(19) United States
(12) Patent Application Publication
(10) Pub. No.: US 2020/0388360 A1
(43) Pub. Date: Dec. 10, 2020

(54) METHODS AND SYSTEMS FOR USING ARTIFICIAL NEURAL NETWORKS TO GENERATE RECOMMENDATIONS FOR INTEGRATED MEDICAL AND SOCIAL SERVICES

(71) Applicant: KONINKLIJKE PHILIPS N.V., Eindhoven (NL)

(72) Inventors: Jennifer CAFFAREL, EINDHOVEN (NL); Gijs GELEIJNSE, GELDROP (NL); Privender Kaur SAINI, VELDHOVEN (NL)

(21) Appl. No.: 17/002,867

(22) Filed: Aug. 26, 2020

Related U.S. Application Data
(63) Continuation-in-part of application No. 14/962,529, filed on Dec. 8, 2015, now abandoned.
(60) Provisional application No. 62/089,892, filed on Dec. 10, 2014.

(51) Int. Cl. G16H 10/60; G06N 3/04; G06N 3/08

(52) U.S. Cl. CPC G16H 10/60; G06N 3/08; G06N 3/04

(57) ABSTRACT

Methods and systems for the use of an artificial neural network to determine the medical and psycho-social needs of patients. Namely, the artificial neural network is trained to predict successful patient care programs based on historical medical and socials outcomes for a plurality of patients, wherein the historical medical and social outcomes are represented in respective vector arrays. For example, in order to prevent bias from being introduced, the artificial neural network is trained on separate vector arrays for medical and social outcomes.

### CROSS-REFERENCE TO RELATED APPLICATION

This application is a continuation-in-part, and claims priority to, U.S. patent application Ser. No. 14/962,529, filed Dec. 8, 2015, which itself claims priority to U.S. Patent App. No. 62/089,872, filed Dec. 10, 2014. Both of these applications are hereby incorporated by reference in their entirety.

### FIELD

The present application relates methods and systems for using artificial neural networks to generate recommendations for integrated medical and social services.

### BACKGROUND

Many chronic disease management programs focus on monitoring and treating a patient's condition while educating and empowering patients (and informal care givers) to take better care of themselves. It is natural to concentrate efforts on the medical needs of the patient. However, a crucial aspect of a patient's care also involves addressing the patient's psychological and social needs.

In clinical practice, medical needs are mainly addressed by the clinical care team. The psychological and social needs of the patient, if addressed, may be reviewed by a healthcare professional (e.g., a discharge nurse, a social worker linked to the hospital, or the like) prior to patient discharge from a hospital. When the disease burden increases for the patient, the need for services addressing the psycho-social status of the patient can increase accordingly. Furthermore, there is general acceptance that addressing the psycho-social needs of a patient positively influences the patient's medical outcome. Unfortunately, the patient's care plan is rarely approached in an integrated and standardized fashion accounting for both medical and psycho-social aspects.

While most healthcare professionals will focus on treating a patient's disease, the patient's ideal treatment plan should help to find the right “disease-life” balance. For example, the patient should learn how to manage their disease such that they can carry out their lives as normally as possible. However, in practice, medical and social services assessment and delivery are often separated, and are rarely integrated to form a holistic care plan for the patient. The delivery of medical services is often well structured and understood (e.g., services provided in several outpatient clinics of one hospital system). For example, medical services are generally well established and are delivered within a fairly structured system (e.g., guidelines and policies that exist to establish eligibility and reimbursement criteria).

Social services, on the other hand, can be provided by official organizations, and/or community/voluntary organizations. The referral, financing, and delivery of social services is much more fragmented and complex. For example, social services can be provided by different service providers that are both formal and informal. In another example, the entitlement and financing aspects in a hospital system can be unclear. While medical services target well-defined, quantifiable outcomes (e.g., readmissions, mortality, and the like), the benefits of social services are generally expressed in more qualitative terms of well-being, patient satisfaction, and quality of life. In addition, the financial budget for the patient is often limited. As a result, these social services may no longer be affordable since the budget is primarily allocated to medical services. A hospitalization or visit to the primary care physician is a good opportunity to evaluate the need for services on a holistic level. Such a discussion may lead to difficult decisions to trade off curative medical services (such as daily home nurse visits) with social services (such as visits from a social worker, a food bank, and the like).

There is a general consensus that both the medical and social needs of a patient should be identified and addressed. However, there is still a lack of coordination and integration in practice. There are several reasons for this, including conservatism, difficulty in assessing social services outcomes, and a lack of knowledge how to integrate medical and social needs.

First, medical sciences and social sciences are often viewed as well-defined and separate. This can lead to a discipline communication gap.

Second, medical health assessments to evaluate a patient's needs are judged to be more concrete, quantitative, and well established, as illustrated by the drive towards evidence-based medicine. There is a lack of consensus on how to assess the social needs of a patient. Social needs are often seen as more qualitative and abstract than medical health needs.

Third, the evidence-based medicine mantra strongly advocates for structured evaluation of medical services. However, the breadth of aspects of a patient's psyche or personal life that social services affect makes it difficult to assess and pinpoint specific and generalizable outcomes.

### SUMMARY

In view of the problems above, methods and systems for using artificial neural networks to generate recommendations for integrated medical and social services are described. For example, due to the breadth of aspects of a patient's psyche or life that social services affect, as well as the number of medical variables that may affect the patient's health, the methods and systems described herein use an artificial neural network to determine the medical and psycho-social needs of patients. The system does this based on historical data regarding medical and social outcomes for a population of patients.

For example, the system identifies patient care programs that exhibit a target medical and/or social outcome on a given population of patients (e.g., patients having a specific category of characteristics), and the system recommends these patient care programs to new patients based on the population categories into which they fall. Importantly, the specific category of characteristics for a patient is based on the results of the medical services applied to the patient, and the results of the social services applied to the patient. For example, the results of the medical services applied to the patient are translated into a medical outcome vector array for the patient, and the results of the social services applied to the patient are translated into a social outcome vector array for the patient. These vector arrays are then used by the artificial neural network to recommend a patient care program.

However, even if the medical outcome vector array and the social outcome vector array are based on the aforementioned criteria, the artificial neural network needs to be trained in a specific way. Namely, the artificial neural network is trained to predict successful patient care programs based on historical medical and socials outcomes for a plurality of patients, wherein the historical medical and social outcomes are represented in respective vector arrays. For example, in order to prevent bias from being introduced, the artificial neural network is trained on separate vector arrays for medical and social outcomes. That is, the artificial neural network is trained to classify labeled patient medical profile vector arrays into corresponding general medical service programs, wherein the general medical service programs each have a targeted medical outcome. Thus, the artificial neural network may predict the general medical service programs having the best medical outcome based on the patient's medical profile (e.g., the inputted medical outcome vector array). The artificial neural network is, likewise, trained to classify labeled patient social profile vector arrays into corresponding general social service programs, wherein the general social service programs each have a targeted social outcome. Thus, the artificial neural network may predict the general social service programs having the best social outcome based on the patient's social profile (e.g., the inputted social outcome vector array).

The artificial neural network may then recommend a patient care program based on the general medical service program, and the general social service program. Through this methodology, bias that results through training the artificial neural network on a single vector array representing both medical and social services is removed. For example, the artificial neural network may use the same weights while working in tandem on two different input vectors to compute comparable output vectors.

In some aspects, methods, and systems of determining medical and psycho-social needs of patients based on historical data on medical and social outcomes using an artificial neural network are described. For example, the system may receive a medical outcome vector array, wherein the medical outcome vector array represents the results of medical services applied to a patient. The system may receive a social outcome vector array, wherein the social outcome vector array represents the results of social services applied to the patient. The system may input the medical outcome vector array and the social outcome vector array into an artificial neural network, wherein the artificial neural network is trained to predict successful patient care programs based on historical medical and social outcomes for a plurality of patients, wherein the historical medical and social outcomes are represented in respective vector arrays. The system may then receive a recommendation from the artificial neural network for a patient care program.

Various other aspects, features, and advantages of the invention will be apparent through the detailed description of the invention, and the drawings attached hereto. It is also to be understood that both the foregoing general description, and the following detailed description are examples, and not restrictive of the scope of the invention. As used in the specification, and in the claims, the singular forms of “a,” “an,” and “the” include plural referents unless the context clearly dictates otherwise. In addition, as used in the specification and the claims, the term “or” means “and/or” unless the context clearly dictates otherwise.

### BRIEF DESCRIPTION OF DRAWINGS

The disclosure may take form in various components and arrangements of components, and in various steps and arrangements of steps. The drawings are only for the purpose of illustrating the preferred embodiments, and are not to be construed as limiting the disclosure.

FIG. 1 is a schematic view showing a patient care plan system in accordance with one aspect of the present disclosure;

FIG. 2 is a chart showing various services and outcomes associated with the patient care plan system of FIG. 1;

FIG. 3 is a schematic view showing multiple components of the patient care plan system of FIG. 1;

FIG. 4 is an exemplary flowchart of one example use of the patient care plan system of FIG. 1;

FIG. 5 is a schematic view showing another example use of the patient care plan system of FIG. 1;

FIG. 6 shows a system featuring a machine learning model configured for use with patient care plan system of FIG. 1; and

FIG. 7 shows graphical representations of artificial neural network models configured for use with the patient care plan system of FIG. 1.

### DETAILED DESCRIPTION

The present disclosure is directed to systems and methods for creating and adjusting a holistic care plan for a patient. As discussed in more detail below, the systems and methods of the present disclosure provide integration of medical and social services into a care plan for a patient. The present disclosure creates a tailored, integrated care plan to address both the medical and psycho-social needs of a patient. Advantageously, the systems and methods of the present disclosure provide a processor that: (1) defines how to identify which services a patient needs based on the patients' general medical, organizational, and psycho-social profile, and accounts for medical, organizational, and financial constraints; (2) matches specific services, or tailors the elements of the services according to a patient's current status and assessment of acuity level/risk in order to maximize outcomes; and (3) integrates medical and social services into a holistic care plan for a patient.

The present disclosure provides new and improved systems and methods which overcome the above-referenced problems and others.

This present disclosure aims to support the creation of a tailored, integrated care plan to address both the medical and psycho-social needs of a patient in two ways. First, the present disclosure provides systems and methods for defining how to identify which categories of patients need which services (e.g., based on population level). This criterion is based on patients' general medical and psycho-social profile, and also considers organizational and financial constraints. Second, the present disclosure provides systems and methods for matching specific services, or tailoring the elements of the services according to a patient's current status and assessment of acuity level/risk in order to maximize outcomes (e.g., based on the individual patient level). The general patient profile and individual status are derived from a holistic patient assessment, which combines the medical and psycho-social needs of the patient.

In accordance with one aspect, a method for creating a patient care plan for a target patient is provided. Inputs related to one or more social services and one or more medical services that are each associated with target patient data are received. One or more social and/or medical services are selected based on a targeted assessment. A net care benefit is calculated for each of the selected services. An outcome patient care plan is created from outcomes including the selected services determined to have the highest net care benefit.

One advantage resides in offering social services to a patient.

Another advantage resides in finding locally available social, including psycho-social, services to a patient.

Another advantage resides in finding a balance between medical treatments and social service treatments.

Another advantage resides in reducing costs while optimizing treatment for a patient.

Still further advantages of the present disclosure will be appreciated to those of ordinary skill in the art upon reading and understanding the following detailed description.

As used herein, the term “service”, and variants thereof, refers to interventions and treatments that exist to treat conditions and/or issues, and to maintain the quality of life of the individual patient. Services can be provided by healthcare professionals (formal healthcare services), social workers, private service providers, charities, or members of the community.

As used herein, the term “outcome,” and variants thereof, refers to a quantifiable result of a clinical and/or a psycho-social service. Outcomes are defined per service, or per group of services.

As used herein, the term “classifier,” and variants thereof, refers to a class or label applied to a concept, such as a type of service. For example, a patient record that is enriched with additional data based on the patient is classified per service type. In another example, a service can be associated with multiple classes or labels.

As used herein, the term “status,” and variants thereof, refers to a quantifiable evaluation of a patient's medical and psycho-social well-being.

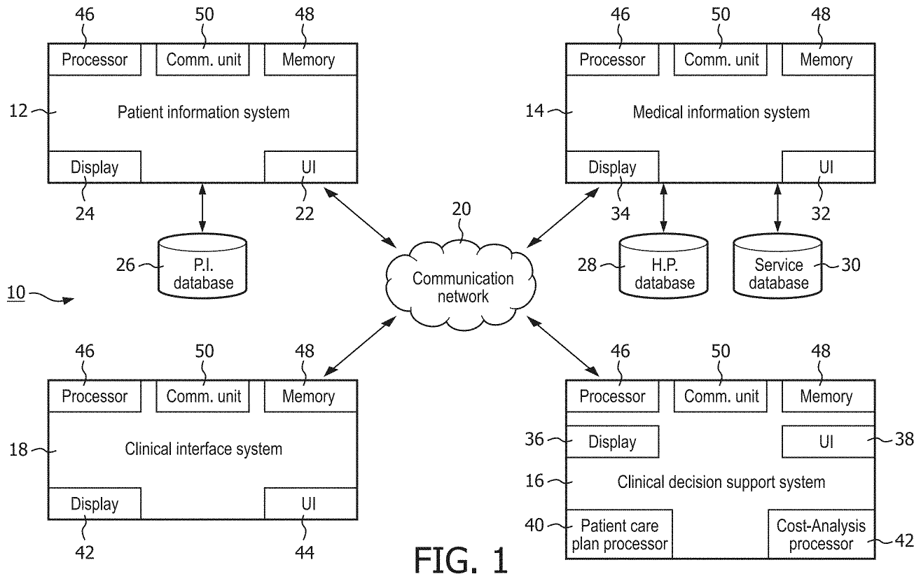

With reference to FIG. 1, a block diagram illustrates one embodiment of a patient care plan system 10 of a medical institution, such as a hospital. The patient care plan system 10 suitably includes a patient information system 12, a medical information system 14, a decision support system (DSS) 16, a clinical interface system 18, and the like, interconnected via a communications network 20. It is contemplated that the communications network 20 includes one or more of the Internet, Intranet, a local area network, a wide area network, a wireless network, a wired network, a cellular network, a data bus, and the like. It should also be appreciated that the components of the patient care plan system 10 can be located at a central location, or at multiple remote locations.

The patient information system 12 stores patient data related to one or more patients being treated by the medical institution. The patient data includes physiological data collected from one or more sensors, laboratory data, imaging data acquired by one or more imaging devices, clinical decision outputs (e.g., early warning scores, state of the patient, etc.), and the like. The patient data may also include the patient's medical records, the patient's administrative data (e.g., patient's name, location, and the like), the patient's clinical problem(s), the patient's demographics, such as weight, age, family history, co-morbidities, and the like. In a preferred embodiment, the patient data includes a unique identifier, medical indication, age, gender, body mass index, systolic/diastolic blood pressure, relevant blood markers, the results of medical questionnaires about the patient's medical and quality of life, and the like. Further, the patient data can be gathered automatically and/or manually. As to the latter, a user input device 22 can be employed. In some embodiments, the patient information system 12 includes one or more display devices 24 that provide users with a user interface within which to manually enter the patient data and/or for displaying generated patient data. In one embodiment, the patient data is stored in the patient information database 26. Examples of patient information systems include, but are not limited to, electronic medical record systems, departmental systems, and the like.

Similarly, the medical information system 14 stores medical data collected from a population that is related to the patient being treated. For example, the medical information system 14 stores population level medical data relating to various clinical problems of differing populations of patients. The medical data includes population level knowledge from literature, retrospective studies, clinical trials, clinical evidence on outcomes and prognosis, and the like. In one embodiment, the medical data includes historical patient data, including the medical indication of patients, the interventions that were prescribed to them, their medical outcomes, and healthcare resource consumption, which is stored in a historical patient database 28. The Health Management System (i.e. operational data specific to an institution, can include medical, psycho-social data, as well as process data) and can include the Patient Information System, and data from individuals and from populations. A Medical Knowledge System (i.e., some form of knowledge repository system) can include data from literature and research databases. The information on past interventions and historical patient database can be based in one or the other with a link (automatic or manual) between the Health Management Systems and the Knowledge Database so that these Intervention and Historical Patient databases would be based on data from the Institution (and/or a combination of data from other sites or sources). In another embodiment, the medical data also includes service data relating to collected medical outcomes and costs for patients who underwent the services of interest, which are stored in a service database 30. Further, the medical data can be gathered automatically and/or manually. To enter the data manually, one or more user input devices 32 can be employed. In some embodiments, the medical information systems 14 include display devices 34 providing users with an interface within which to manually enter the medical data, and/or for displaying generated medical data. Examples of medical information systems include, but are not limited to, medical literature databases, medical trial and research databases, regional and national medical systems, and the like.

The DSS 16 stores clinical models and algorithms embodying the clinical support tools or patient decisions aids. The clinical models and algorithms typically include one or more suggested or entered diagnosis and/or treatment options/orders as a function of the patient data and the clinical problem of the patient being treated. Further, the clinical models and algorithms typically generate medical, lifestyle, and/or psycho-social data that includes one or more interventions for the various diagnosis and/or treatment options, and the clinical context based on the state of the patient and the patient data. Specifically, the clinical models and/or guidelines are determined from the diagnoses and/or treatment orders for patients with specific diseases or conditions, and are based on the best available evidence, (i.e., based on clinical evidence acquired through scientific method and studies, such as randomized clinical trials). After receiving patient data, the DSS 16 applies the clinical model and algorithm pertinent to the clinical problem of the patient being treated, and generates medical data including one or more services for the various diagnosis and/or treatment options. It should also be contemplated that as more patient data becomes available, the DSS 16 updates the medical data, including one or more services for the diagnosis and/or treatment options available to the patient. The DSS 16 includes a display 36 (such as a CRT display, a liquid crystal display, a light emitting diode display, and the like) to display the clinical models and algorithms, and a user input device 38, such as a keyboard and mouse, for the clinician to input and/or modify the clinical models and algorithms. The DSS 16 further includes a patient care plan processor 40, and a cost analysis processor 42, as described in more detail below.

The clinical interface system 18 enables the user to input the patient values, lifestyle regimes, ability-to-pay, and preferences related to diagnosis and treatment from a patient's perspective, which are used to select the most cost-effective service for a specific patient from multiple service programs applicable to that patient's clinical condition. In one embodiment, the clinical interface system 18 enables the user to enter specific settings for the cost-effectiveness analysis. These settings may include time horizons for the analysis, discount rates for effects and costs, and ability-to-pay. The clinical interface system 18 also receives a quantitative evaluation and comparison of the alternative choices of services to the patient (not shown) being treated in the medical institution. For example, the clinical interface system 18 displays the quantitative evaluation and comparison of the choices of services, including a comparison of alternative choices on the same measure, such as allowing the patients to adjust for lifestyle regime and preferences, outcome parameters, patient pathways, a desired probability of an overall outcome, or of a specific outcome parameter, and the like, including the cost effects of those choices. The clinical interface system 18 includes a display 42 (such as a CRT display, a liquid crystal display, a light emitting diode display, and the like) to display the evaluation and/or comparison of choices and a user input device 44 such as a keyboard and a mouse, for the user to input the patient values and preferences and/or modify the evaluation and/or comparison. Examples of clinical interface systems 18 include, but are not limited to, a software application that could be accessed and/or displayed on a personal computer, web-based applications, tablets, mobile devices, cellular phones, and the like.

The components of the patient care plan system 10 suitably include processors 46 executing computer executable instructions embodying the foregoing functionality, where the computer executable instructions are stored on memories 48 associated with the processors 46. It is, however, contemplated that at least some of the foregoing functionality can be implemented in hardware without the use of processors. For example, analog circuitry can be employed. Further, the components of the patient care plan system 10 include communication units 50 providing the processors 46 with an interface from which to communicate over the communications network 20. Even more, although the foregoing components of the patient care plan system 10 were discretely described, it is to be appreciated that the components can be combined.

The DSS 16 also selects the most cost-effective intervention or treatment for a specific patient from multiple interventions or treatment programs applicable to that patient's clinical condition.

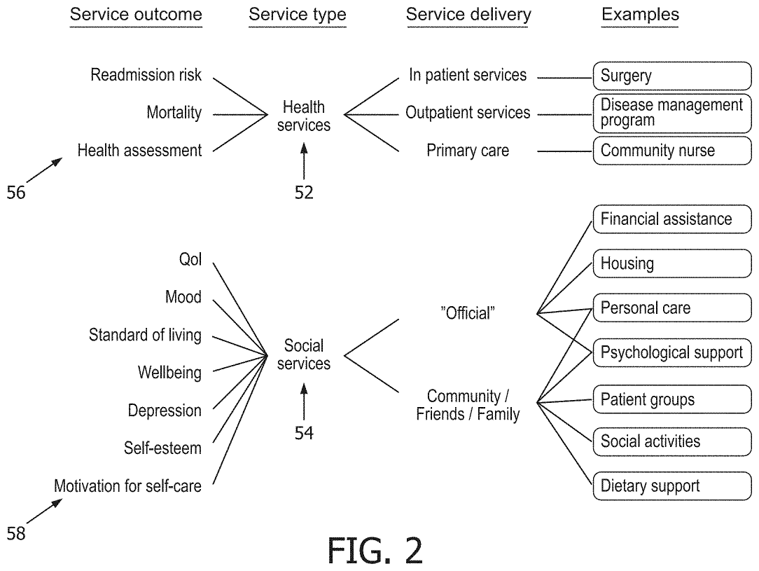

In one example, a plurality of input health services 52 (or medical services) and a plurality of input social services 54 are stored in the service database 30. With reference to FIG. 2, the plurality of health services 52 are each associated with a plurality of medical service outcomes 56, and the plurality of social services 54 are each associated with a plurality of social service outcomes 58. In one example, the health services 52 are selected from a group that includes inpatient services (e.g., surgery and the like), specialized outpatient services (e.g., disease management programs and the like), primary care (e.g., a community nurse and the like), rehabilitation services (e.g., physical therapy and the like), mental medical services (e.g., psychiatry and the like), and palliative care (e.g., preventative care and the like). The medical service outcomes 56 include readmission risk, mortality, and medical assessment of the patient, among others.

In another example, the social services 54 can include both social care services and community resources. The social services 54 are selected from a group that includes financial assistance services, housing services, personal care services, psychological support services, patient group services, social activities services, dietary support services, employment services, income services, legal services, transportation and mobility services, care management services, home care services, information and assistance services, long-term care services, nutrition and meals services, respite services, senior services, volunteer and intergenerational services, and wellness and well-being services. The social service outcomes 58 include quality of life, patient mood, standard of living, wellbeing, depression, self-esteem, motivation for self-care, and patient engagement, among others. For example, if the health service 52 is telemedicine monitoring, the medical service outcome 56 includes days without hospitalization, drug therapy adherence (e.g., measured by number of intake moments missed); blood pressure levels; status of self-reported symptoms (e.g., pain level) and the like. In another example, if the social service 54 is a food delivery program, the social service outcome 58 includes body weight, reported satisfaction, and the like.

The selected services 52 and 54 are cross-checked to ensure that they optimally address the mix of psycho-social determinants and clinical needs. For example, the selected services 52 and 54 are associated with a confidence score that indicates the applicability of the services to a target case. These confidence scores are based on evidence of services 52 or 54 prescribed or arranged for similar past target cases. By doing so, potentially overlapping and/or conflicting services are highlighted such that there is no redundancy in the patient's care plan. As a result, the effectiveness and cost of the selected services 52 and 54 are balanced to see if similar needs are being addressed. For example, it is important to flag a health service 52 which would provide a good medical outcome, but a lower quality of life due to intense daily clinic visits, especially if one of the aims of the care plan is to address the quality of life via a selected social service 54. A user can tailor the social service 54 to compensate for the additional loss in the social outcome 58 (e.g., quality of life) or tailor the selected health service 52 by seeking other ways to deliver it (e.g., ambulatory or home care).

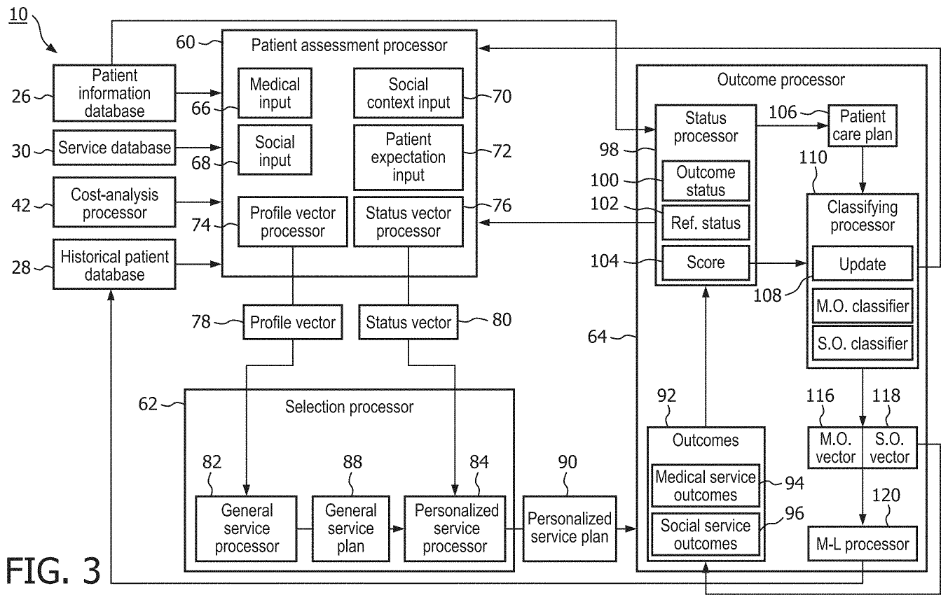

With reference to FIG. 3, the DSS also selects the most cost-effective intervention or treatment for a specific patient from multiple service programs applicable to that patient's clinical condition. Specifically, the DSS 16 includes the patient care plan processor 40 which generates a patient care plan utilizing the medical data stored in a medical information system 14. The patient care plan processor generates a patient care plan model in the form of a support vector array machine, Bayesian classifier, or any other statistical model that can be used to associate cases with outcomes and recommendations for interventions or services. Other applicable prediction models will be known to those skilled in the art. The generation of the patient care plan is described in more detail below.

The cost analysis processor 42 retrieves detailed patient data for a specific patient, from the patient information system and utilizes the patient care plan, the one or more services for the various diagnosis and/or treatment options, and medical data from the medical information system and information on insurance or financial constraints to calculate predictions for the medical and cost outcomes of the patient. For example, the cost analysis processor 42 receives predictions of medical outcomes and healthcare resource consumption to estimate costs and effects of the services of interest specific to the specific patient. The cost analysis processor 42 generates displays for the estimated costs and effects specific to the patient for each intervention. Based on the result of comparing costs and effects, a recommendation for the most cost-effective intervention for the patient is displayed on the clinical interface system.

Specifically, the cost analysis processor 42 retrieves relevant patient data from the patient information system 12 that are utilized in the prediction model including name, medical indication, age, gender, body mass index, systolic/diastolic blood pressure and values of blood markers specific to the medical indication. The cost analysis processor 42 utilizes the patient care plan to generate medical outcomes, such as estimated survival rates (projected or estimated) and hospital admission rates. These rates are then further used by the cost analysis processor 42 to compute effects and costs over a given time horizon for each intervention. Costs are subtracted from the gross medical effects after adjusting the costs by a so-called “ability-to-pay” value (e.g., the amount of money the patient is able and/or willing to pay for the services). For each service, this results in a value with a unit equal to the medical effects, called the “net medical benefits”. The service with the highest net medical benefits is then recommended to the user.

In another example, the cost analysis processor 42 predicts the patient-specific medical, social, and economic outcomes (e.g., disease-related risks or hazards for a target patient) based on results from retrospective data analysis of the patient, outcome, and cost data. The outcomes are then combined in a cost analysis to establish the most cost-effective service for the patient. Different intervention or treatments are compared using a quantity known as the “net medical benefits”, which include medical outcomes (weighted by quality of life), expected costs, as well as the ability-to-pay (amount able to invest for the service). A recommendation of this service is provided to the user via the clinical interface system 18.

In another example, the cost analysis processor 42 couples the direct cost with estimated patient risks by using time integrals, correct for quality of life, and performs a cost analysis for each service strategy. These estimations allow for comparison between service strategies on risk (estimated outcome), direct costs (accumulated over time, given the risks) and cost-effects, to be varied over different time horizons (30-days, one-year, life-time, and the like). A ranked list or a single recommendation of the most cost-effective service strategies can then be provided to the decision maker (e.g., clinical specialist) or the patient via the clinical interface system 18.

In another embodiment, the cost analysis processor 42 provides the net medical benefits change as a function of the ability-to-pay. The net medical benefits of selected services can be visualized to the user as a function of the ability-to-pay. This analysis is used to indicate if a single service is always dominating other services (i.e., the net medical benefits are always higher for this service, regardless of the ability-to-pay). It may also be indicated if a combination of multiple services are dominating other services over the entire range of ability-to-pay values (e.g., service 1 results in the highest net medical benefits for ability-to-pay values below “X”, and service 2 results in the highest net medical benefits for ability-to-pay values above “X.”)

In a further embodiment, the cost analysis processor 42 provides a cost analysis for two or more services for a patient population. For example, the net medical benefits may be aggregated over multiple patients who, given their medical condition, are eligible for the same services. This information may be used to recommend a service for a population of patients. The patient population is derived from both medical and social aspects.

The patient care plan processor 40 is associated with each of the patient information database 26, the historical patient database 28, and the cost analysis processor 42. The patient care plan processor 40 includes an assessment processor 60, a selection processor 62, and an outcome processor 64. The patient care plan processor 40 includes an assessment processor 60 that assesses a target patient's profile to select focus needs and related outcomes for the target patient. This assessment is expressed through clinical and psycho-social aspects. For example, the assessment processor 60 receives and assesses medical inputs 66 (e.g., heart conditions, respiratory conditions, illnesses, and the like), social inputs 68 (dementia, anger issues, lack of coping skills, and the like), social context inputs 70 (e.g., marital status, children, pets, relatives, friends, etc.), and patient expectation inputs 72 (e.g., living at home, assisted living, regain dexterity, regain movement, etc.).

A profile vector array processor 74 and a status vector array processor 76 of the assessment processor 60 uses the inputs 66, 68, 70, and 72 to generate vector arrays relating to the inputs. In one example, the profile vector array processor 74 generates a profile 78, such as a profile vector array, of the patient using the inputs 66, 68, 70, and 72. The profile vector array processor 74 includes an input-to-vector array mapping process to generate the profile vector array 78. Specifically, the profile vector array processor 74 extracts context information values of each input 66, 68, 70, and 72 and converts these values to vector array values of the profile vector array 78. For example, each input 66, 68, 70, and 72 is input into a pre-calculated lookup table, a neural network, or the like. Subsequently, the profile vector array 78 is used to determine the optimal service arrangement to serve the selected psycho-social determinants and/or the selected clinical outcomes, as described in more detail below.

In another example, the status vector array processor 76 generates a status 80, such as a status vector array, of the target patient using the inputs 66, 68, 70, and 72. The status vector array processor 76 includes an input-to-vector array mapping process to generate the status vector array 80. Specifically, the status vector array processor 76 extracts context information values of each input 66, 68, 70, and 72 and converts these values to vector array values of the status vector array 80. For example, each input 66, 68, 70, and 72 is input into a pre-calculated lookup table, a neural network, or the like. Subsequently, the status vector array 80 is used to determine the optimal service arrangement to serve the selected psycho-social determinants and/or the selected clinical outcomes, as described in more detail below.

The selection processor 62 selects which of the health services 52 and the social services 54 would benefit the target patient based on the profile vector array 74 and the status vector array 80. In other words, the selection processor 62 selects the health services 52 and the social services 54 that have the best effect on both the medical and well-being of the target patient. For example, the selection processor 62 provides: (1) a general view of the patient's needs, and uses clustering methods to allocate services 52 and 54 on a generic population level; and (2) a specific view of the patient's needs to allocate services 52 and 54 solely for the target patient. To do so, the selection processor 62 includes a general service processor 82 and a personalized service processor 84.

The general service processor 82 is programmed to select generic health services and generic social services based on the profile vector array For example, the assessment processor 60 transfers the profile vector array 74 after generation thereof to the general service processor 82. The general service processor 82 also receives information from the patient input database 26 (e.g., patient data), the historical patient database 28 (e.g., medical data collected from a population that is related to the patient), and the cost-analysis processor 42 (e.g., ability of the patient to pay for the health services 52 and/or the social services 54). The general service processor 82 includes a data-mining processor 86 that is programmed to search the service database 30 for health services 52 and/or social services 54 that correspond to the profile vector array 78, the patient data from the patient input database 26, the population data from the historical patient database 28, and the net health benefit analysis from the cost-analysis processor 42. From this information, the general service processor 82 creates a general service care plan 88 that includes the selected health services 52 and the selected social services 54 that correspond to the needs of the population with similar profile vector arrays 78 to that of the target patient.

The personalized service processor 84 is programmed to select personalized health services 52 and personalized social services 54 based on the status vector array 80. For example, the assessment processor 60 transfers a personalized status vector array 80 after generation thereof to the personalized service processor 84. The personalized service processor 84 also receives the general service care plan 88 from the general service processor 82. The personalized service processor 84 includes a data-mining processor that is programmed to search the general service care plan 88 for health services 52 and/or social services 54 that correspond to the status vector array 80. The personalized service processor 84 creates a personalized service plan 90 that includes the selected health services 52 and the selected social services 54 that correspond to the personalized needs of the target patient based on the status vector array 80 thereof.

In another example, the outcome processor 64 is programmed to continuously monitor and evaluate the outcomes 92 of the personalized service plan 90. To do so, the outcome processor 64 receives the personalized service plan 90 from the personalized service processor 82 and simulates the personalized service plan 90 to predict the outcomes 92. The outcomes 92 include medical service outcomes 94 and social service outcomes 96.

The outcome processor 64 includes a status processor 98 that identifies and classifies the medical services outcomes 94 and the social service outcomes 96. Each outcome 92 is quantified on a scale of 0 (very unfavorable) to 100 (most favorable). For each outcome 92, the status processor 98 computes an outcome status 100 for each possible outcome 92 that is based on the personalized service plan 90 in combination with measurements from patient monitors, usage of devices, recent clinical data, and other known information to assess the target patient on the various outcomes statuses 100. The outcome statuses 100 define the actual current assessment of the target patient, and are transferred to the patient assessment processor 60, as described in more detail below.

In another example, the status processor 98 is programmed to compute a reference status 102 for each outcome 92 for a population of patients similar to the target patient. To do so, the status processor 98 computes multiple outcome statuses 100 for multiple patients that correspond to the needs of the population with similar profile vector arrays 78 to that of the target patient. The reference statuses 102, once computed, are transferred to the historical patient database as described in more detail below.

In a further example, the status processor 98 is programmed to compute a score 104 for each outcome 92. To do so, the outcome status processor 98 subtracts the reference statuses 102 from the outcome statuses 100 for each outcome 92. In one example, a positive score 104 indicates that the expected outcome 92 is better for a population of patients similar to the patient (e.g., the target patient does not need a service 52 or 54 for the outcome 92). In another example, a negative score 104 indicates that the expected outcome 92 is worse for the population of patients similar to the patient (e.g., the target patient needs a service 52 or 54 for the outcome 92). The scores 104, once computed, are transferred back to the general service processor 82, as described in more detail below. Once the scores 104 have been calculated, the selected medical service outcomes 94 and social service outcomes 96 are used to create a patient care plan 106. The services 52 and 54 are then installed in the target patient's house.

The status processor 98 is programmed to produce self-effectiveness evaluation updates 108 of the patient care plan 106. To do so, the scores 104 are collected into a classifying processor 110 of the status processor 98 for sorting the outcomes 92. The classifying processor 110 can include a medical outcome classifier and a social outcome classifier. In one example, the medical outcome classifier 112 and the social outcome classifier 114 generate medical outcome vector arrays 116 and social outcome vector arrays 118, respectively, that each include a label. The medical outcome classifier 112 and the social outcome classifier 114 each include an input-to-vector array mapping process to generate the medical outcome vector arrays 116 and the social outcome vector arrays 118. Specifically, the medical outcome classifier 112 and a social outcome classifier 114 extract context information values of each score 104, converts these values to vector array values of the medical outcome vector array 116, and the social outcome vector array 118. For example, each score 104 is input into a pre-calculated lookup table, input into a neural network, or the like.

The medical outcome vector array 116 and the social outcome vector array 118 are compared to similar vector arrays contained within the historical patient database 28 using a machine-learning processor 120 (e.g., K-nearest neighbors, support vector array machines, decision tree learning, support vector array machines, neural networks, inductive logic programming, clustering, association rule learning, Bayesian networks, reinforcement learning, representation learning, similarity learning, sparse dictionary learning, and the like). For example, a social service 54 of “meal services” is included in a previously obtained social outcome vector array 118 that is compared to the social outcome vector array 118 for the target patient. The medical outcome vector array 116 and the social outcome vector array 118 are then calculated for each medical service 52 and each social service 54. Consequently, the patient care plan 106 can be updated and improved based on patient care plans for other patients similar to the target patient. The evaluation updates 108, once computed, are recirculated into the status processor 98 to further refine the patient care plan 106.

In addition, the outcomes statuses 100, the references statuses 102, and the scores 104 are similarly inputted back into the patient assessment processor 60, the historical patient database 28, and the general service processor 82, respectively, to continuously update the patient care plan 106. For example, the selected outcomes 92 of the patient care plan 106 are revisited for the mortality score 104. The mortality score 104 is used to balance the focus between health services 52 and the social services 54. In other words, the higher the chance of early mortality, the more budget is allocated for the social services 54 via the cost analysis processor 42. The cost analysis processor 42 re-computes the balance between the health services 52 and social services 54. The services 52 and 54 are selected that optimize the selected outcomes 92 within the service and clinical budget.

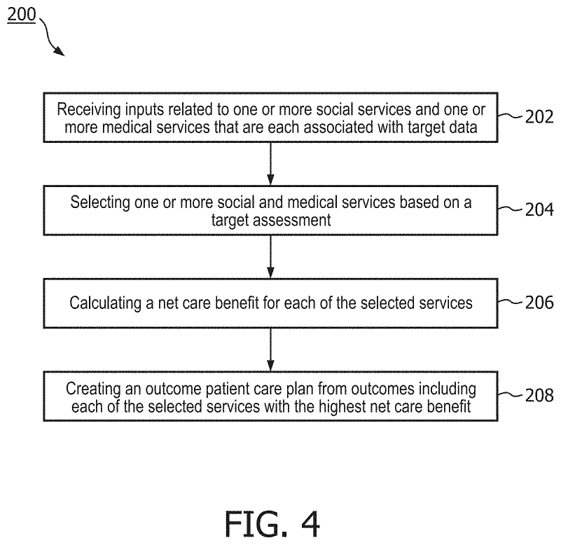

With reference to FIG. 4, a method 200 for creating a patient care plan is provided. At Step 202, inputs 66, 68, 70, 72 related to one or more social services 54 and one or more health services 52 that are each associated with target data are received. At Step 204, one or more social and health services 52, 54 are selected based on a target assessment. At Step 206, a net care benefit is calculated for each of the selected services 52, 54. At Step 208, the patient care plan 106 is created from outcomes 92, including each of the selected services 52, 54 with the highest net care benefit.

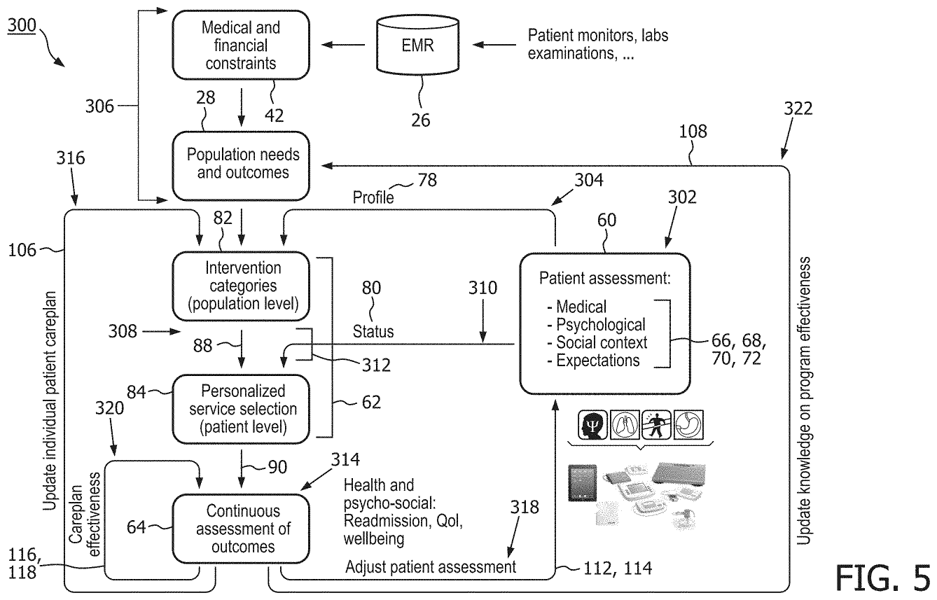

With reference to FIG. 5, a method 300 is provided for updating the individual patient care plan 106. At Step 302, inputs 66, 68, 70, 72 relate to one or more social services 54 and one or more health services 52 that are each associated with target data and are received in the assessment processor 60. At Step 304, the profile vector array 78 is generated. At Step 306, data from the patient database 26, the historical patient database 28, the cost analysis processor 42, and the profile vector array 78 are received by the general service processor 82. At Step 308, the generalized service plan 88 is generated. At Step 310, the status vector array 80 is generated. At Step 312, the generalized service plan 88 and the status vector array 80 are received by the personalized service processor 84. At Step 314, the personalized service plan 90 is generated and transferred to the outcome processor 64. At Step 316, the patient care plan 106 is generated from the scores 104 of the personalized service plan 90. At Step 318, the medical and social service outcome classifiers 112 and 114 are generated and transferred to the assessment processor 60. At Step 320, the medical and social outcome vector arrays 116 and 118 are generated and transferred to the outcome status processor 98. At Step 322, the evaluation updates 108 are generated and transferred to the historical patient database 28.

### Example

In one example of the method 200 or 300, a 75-year-old male patient (“the patient”) is suffering from heart failure, chronic obstructive pulmonary disease (“COPD”), and has difficulties managing his temper and has short-term memory problems. He is a widower and lives only with a dog. He is hospitalized for pneumonia. He wishes to return home and live relatively independently for the next 10 years.

The patient's medical conditions are inputted into the patient input database 26, and his cost conditions are input into the cost analysis processor 42 by a multi-disciplinary team of medical and social care professionals. The assessment processor 60 receives and assesses the data from the patient input database 26. For example, the assessment processor 60 sorts the data from the patient input database 26 into medical inputs 66 (e.g., heart failure, COPD, pneumonia), social inputs 68 (e.g., onset dementia, lack of anger coping skills), social context inputs 70 (e.g., widowed, lives with a dog), and patient expectation inputs 72 (e.g., return home, live independently for the next 10 years). From the inputs 66, 68, 70, and 72, the profile vector array processor 74 generates the profile vector array 78 and the status processor 76 generates the status vector array 80.

The selection processor 64 then selects which of the health services 52 and the social services 54 from the service database 30 that would benefit the patient based on the profile vector array 74 and the status vector array 80. The general service processor 82 creates the general service care plan 88 that includes the selected health services 52 and the selected social services 54 that correspond to the needs of the population with similar profile vector arrays 78 to that of the patient. For example, the selected general health services 52 include pulmonary rehabilitation service upon discharge and physical activity. The selected general social services 54 include temporary home care (e.g., a home medical care worker to provide services hygiene, food, logistics, and the like), nutritional services, psychological care, neurological consultations, and the like.

The personalized service processor 84 creates the personalized service plan 90 that includes the selected health services 52 and the selected social services 54 that correspond to the personalized needs of the patient based on the status vector array 80 thereof. For example, the personalized health services 52 include: (1) a pulmonary rehabilitation service that can be held at a local outpatient clinic; be an individualized session; and can be scheduled on desired days of the week; and (2) a physical activity plan that includes regular dog walks; and walks that can be adjusted to account for the patient's heart failure and COPD. In another example, the personalized social services 54 include: (1) a temporary home care service for 2 weeks; (2) a nutritional intervention service that includes consultation with a dietitian, is salt-free (due to the patient's heart failure) and is a high-protein diet (due to the patient's COPD); psychological care that includes anger management classes; neurological consultations that include a postponement until the patient determines that his insurance covers the service; and a recommendation that the patient be closely monitored to follow the dementia progression; and a recommendation to be put on a waiting list for an independent senior living facility; and a pet care charity that can take care of the patient's dog during hospital visitations.

The outcome processor 64 receives the personalized service plan 90 from the personalized service processor 82 and simulates the personalized service plan 90 to produce the medical service outcomes 94 and the social service (including psycho-social) outcomes 96. The status processor 98 identifies and classifies the medical services outcomes 94 and the social service outcomes 96. The status processor 98 computes the score 104 for each outcome 92. For example, the following services listed are those that have the highest net medical benefit. The medical service outcomes 94 include: (1) a pulmonary rehabilitation service that includes a six minute walking time and a focus on lung capacity, a focus on exercise load, a questionnaire with questions relating to the daily living activities of the patient, and the number of cigarettes smoked per day by the patient; and (2) the physical activity plan that includes a step count plan and an adherence to activity plan. The social service outcomes 96 include: (1) a temporary home care outcome with a focus on increasing patient independence; (2) a nutritional service outcome with a focus on weight, fat mass, and blood test of the patient; (3) neurological consultations with a focus of a prognosis for the progression of the patient's dementia; and (4) a psychological care outcome with a focus on patient satisfaction, an anxiety depression scale, an evaluation of the patient by an expert, and an adherence to the anger management session. The selected outcomes 92 constitute the patient care plan 106.

The status processor 98 produces self-effectiveness evaluation updates 108 of the patient care plan 106. To do so, the scores 104 are collected by the medical outcome classifier 112 and the social outcome classifier 114. The medical outcome vector array 116 and the social outcome vector array 118 are compared to similar vector arrays contained within the historical patient database 28 using the machine-learning processor 120. In this example, upon a self-evaluation update 108, the scores 104 of the outcomes 92 of the patient care plan 106 are determined to have positive scores 104 (i.e., the patient has improved) except for the anger management outcome (i.e., the patient has not improved). For example, the status vector array 80 is updated to state that the patient's heart failure is stable, the COPD is controlled, and the patient no longer has pneumonia, but that the patient's coping skills did not improve. As a result, the patient medical care plan 106 is updated to discharge the patient from all other services 52 and 54, except for the psychological care service (e.g., anger management classes, waiting list independent for senior living).

The patient care plan system 10 is connected to a wide range of sensors and other input data sources. For example, the patient care plan system 10 can be connected to a hospital's information system (e.g., that includes the patient's current and past medical status), other diagnostic data sources (e.g., lab information systems, pharmacy records, monitoring of vital signs, and the like), home medical devices (e.g., weight scales, blood pressure devices, CPAP devices, nebulizers, and the like), home devices (e.g., tablets, television, activity monitors, and the like), questionnaires where patients report their symptoms, attitudes and beliefs, a database describing the patient's current social and clinical services, a database describing the patient's financial constraints and budget for services. The data monitored through the combination of services 52 and 54 is fed back into the method to re-assess the services.

As used herein, a memory includes one or more of a non-transient computer readable medium; a magnetic disk or other magnetic storage medium; an optical disk or other optical storage medium; a random access memory (RAM), read-only memory (ROM), or other electronic memory device or chip or set of operatively interconnected chips; an Internet/Intranet server from which the stored instructions may be retrieved via the Internet/Intranet or a local area network; or so forth. Further, as used herein, a processor includes one or more of a microprocessor, a microcontroller, a graphic processing unit (GPU), an application-specific integrated circuit (ASIC), a field-programmable gate array (FPGA), personal data assistant (PDA), cellular smartphones, mobile watches, computing glass, and similar body worn, implanted or carried mobile gear; a user input device includes one or more of a mouse, a keyboard, a touch screen display, one or more buttons, one or more switches, one or more toggles, and the like; and a display device includes one or more of a LCD display, an LED display, a plasma display, a projection display, a touch screen display, and the like. Stated another way, the patient care plan system 10 can be a non-transitory computer readable medium carrying software to control a processor.

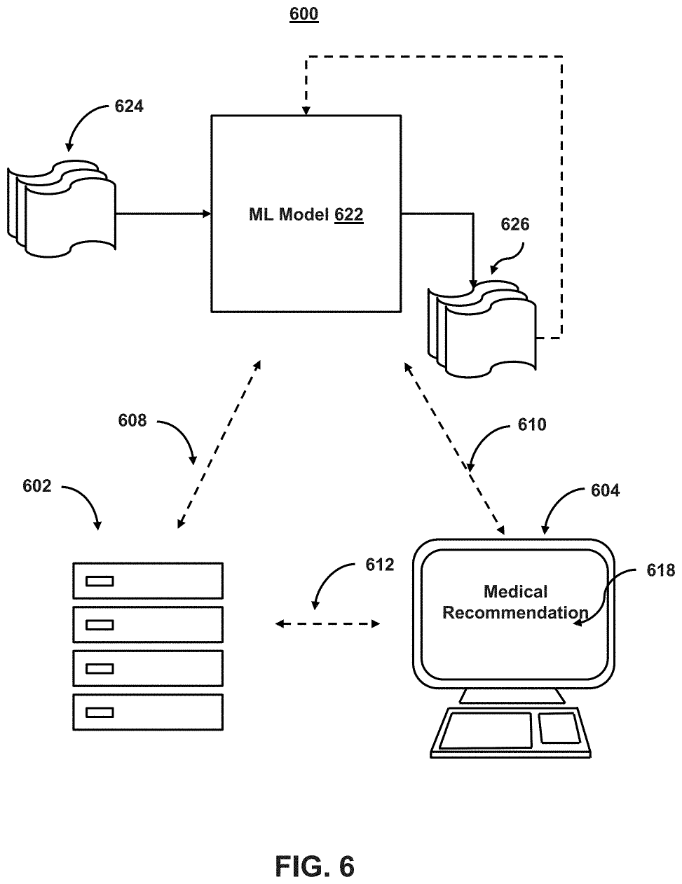

FIG. 6 shows a system featuring a machine learning model configured for use with patient care plan system of FIG. 1. As shown in FIG. 6, system 600 may include client device 602, client device 604 or other components. Each of client devices 602 and 604 may include any type of mobile terminal, fixed terminal, or other device. Each of these devices may receive content and data via input/output (hereinafter “I/O”) paths and may also include processors and/or control circuitry to send and receive commands, requests, and other suitable data using the I/O paths. The control circuitry may comprise any suitable processing circuitry. Each of these devices may also include a user input interface and/or display for use in receiving and displaying data. By way of example, client devices 602 and 604 may include a desktop computer, a server, or other client device. Users may, for instance, utilize one or more client devices 602 and 604 to interact with one another, one or more servers, or other components of system 600. It should be noted that, while one or more operations are described herein as being performed by particular components of system 600, those operations may, in some embodiments, be performed by other components of system 600. As an example, while one or more operations are described herein as being performed by components of client device 602, those operations may, in some embodiments, be performed by components of client device 604. It should be noted that, although some embodiments are described herein with respect to machine learning models, other prediction models (e.g., statistical models or other analytics models) may be used in lieu of or in addition to machine learning models in other embodiments (e.g., a statistical model replacing a machine learning model and a non-statistical model replacing a non-machine-learning model in one or more embodiments).

Each of these devices may also include memory in the form of electronic storage. The electronic storage may include non-transitory storage media that electronically stores information. The electronic storage media of the electronic storages may include one or both of (i) system storage that is provided integrally (e.g., substantially non-removable) with servers or client devices or (ii) removable storage that is removably connectable to the servers or client devices via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storage may store software algorithms, information determined by the processors, information obtained from servers, information obtained from client devices, or other information that enables the functionality as described herein.

FIG. 6 also includes communication paths 608, 610, and 612. Communication paths 608, 610, and 612 may include the Internet, a mobile phone network, a mobile voice or data network (e.g., a 4G or LTE network), a cable network, a public switched telephone network, or other types of communications network or combinations of communications networks. Communication paths 608, 610, and 612 may separately or together include one or more communication paths, such as a satellite path, a fiber-optic path, a cable path, a path that supports Internet communications (e.g., IPTV), free-space connections (e.g., for broadcast or other wireless signals), or any other suitable wired or wireless communication path or combination of such paths. The computing devices may include additional communication paths linking a plurality of hardware, software, and/or firmware components operating together. For example, the computing devices may be implemented by a cloud of computing platforms operating together as the computing devices.

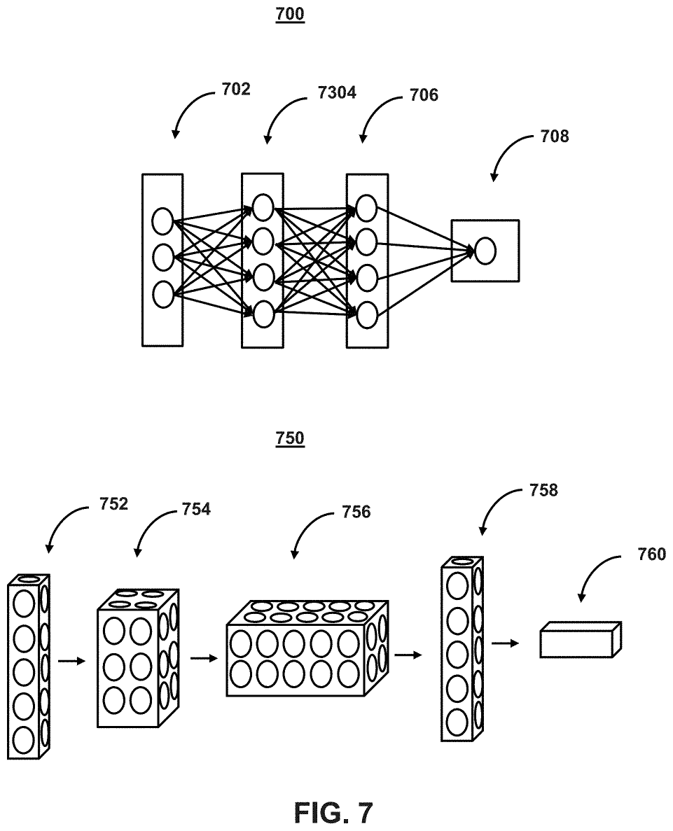

In some embodiments, system 600 may use one or more prediction models to determine medical and psycho-social needs of patients based on historical data on medical and social outcomes using an artificial neural network. For example, as shown in FIG. 6, system 600 may generate a recommendation for a patient care plan using machine learning model 622. The determination may be shown as output 618 on a client device 604. The system may include one or more neural networks (e.g., as discussed in relation to FIG. 7) or other machine learning models. For example, the artificial neural network may use the same weights while working in tandem on two different input vectors to compute comparable output vectors.

As an example, with respect to FIG. 6, machine learning model 722 may take inputs 724 and provide outputs 726. The inputs may include multiple data sets such as a training data set and a test data set. The data sets may represent historical and social outcomes. In one use case, outputs 726 may be fed back to the machine learning model 722 as input to train the machine learning model 722 (e.g., alone or in conjunction with user indications of the accuracy of outputs 726, labels associated with the inputs, or with other reference feedback information). In another use case, machine learning model 722 may update its configurations (e.g., weights, biases, or other parameters) based on its assessment of its prediction (e.g., outputs 726) and reference feedback information (e.g., user indication of accuracy, reference labels, or other information). In another use case, where machine learning model 722 is a neural network, connection weights may be adjusted to reconcile differences between the neural network's prediction and the reference feedback. In a further use case, one or more neurons (or nodes) of the neural network may require that their respective errors be sent backward through the neural network to facilitate the update process (e.g., backpropagation of error). Updates to the connection weights may, for example, be reflective of the magnitude of error propagated backward after a forward pass has been completed. In this way, for example, the machine learning model 722 may be trained to generate better predictions. The machine learning model 722 may be trained to predict successful patient care plans based on medical history and social outcomes for a plurality of patients, wherein the historical and social outcomes are represented in respective vector arrays.

FIG. 7 shows graphical representations of artificial neural network models configured for use with the patient care plan system of FIG. 1. Model 700 illustrates an artificial neural network. Model 700 includes input level 702. Medical outcome vector arrays and social outcome vector arrays can be entered into model 700 at this level. Model 700 also includes one or more hidden layers (e.g., hidden layer 704 and hidden layer 706). Model 700 may be based on a large collection of neural units (or artificial neurons). Model 700 loosely mimics the manner in which a biological brain works (e.g., via large clusters of biological neurons connected by axons). Each neural unit of a model 700 may be connected with many other neural units of model 700. Such connections can be enforcing or inhibitory in their effect on the activation state of connected neural units. In some embodiments, each individual neural unit may have a summation function which combines the values of all of its inputs together. In some embodiments, each connection (or the neural unit itself) may have a threshold function, such that the signal must surpass, before it propagates to other neural units. Model 700 may be self-learning and trained, rather than explicitly programmed, and can perform significantly better in certain areas of problem solving, as compared to traditional computer programs. During training, output layer 708 may correspond to a classification of model 700 and an input known to correspond to that classification may be input into input layer 702. In some embodiments, model 700 may include multiple layers (e.g., where a signal path traverses from front layers to back layers). In some embodiments, back propagation techniques may be utilized by model 700 where forward stimulation is used to reset weights on the “front” neural units. In some embodiments, stimulation, and inhibition for model 700 may be freer flowing, with connections interacting in a more chaotic and complex fashion. Model 700 also includes output layer 708. During testing, output layer 708 may indicate whether or not a given input corresponds to a classification of model 700.

FIG. 7 also includes model 750, which is a convolutional neural network. The convolutional neural network is an artificial neural network that features one or more convolutional layers. Convolution layers extract features from an input image. Convolution preserves the relationship between pixels by learning features using small squares of input data. As shown in model 750, input layer 752 may proceed to convolution blocks 754 and 756 before being output to convolutional output 758. In some embodiments, model 750 may itself serve as an input to model 700.

In some embodiments, model 750 may implement an inverted residual structure where the input and output of a residual block (e.g., block 754) are thin bottleneck layers. A residual layer may feed into the next layer and directly into layers that are one or more layers downstream. A bottleneck layer (e.g., block 758) is a layer that contains few neural units compared to the previous layers. Model 750 may use a bottleneck layer to obtain a representation of the input with reduced dimensionality. An example of this is the use of autoencoders with bottleneck layers for nonlinear dimensionality reduction. Additionally, model 750 may remove non-linearities in a narrow layer (e.g., block 758) in order to maintain representational power. In some embodiments, the design of model 750 may also be guided by the metric of computation complexity (e.g., the number of floating-point operations). In some embodiments, model 750 may increase the feature map dimension at all units to involve as many locations as possible instead of sharply increasing the feature map dimensions at neural units that perform down sampling. In some embodiments, model 750 may decrease the depth and increase the width of residual layers in the downstream direction.

The invention has been described with reference to the preferred embodiments. Modifications and alterations may occur to others upon reading and understanding the preceding detailed description. It is intended that the invention be constructed to include all such modifications and alterations insofar as they come within the scope of the appended claims or the equivalents thereof.

The present techniques will be better understood with reference to the following enumerated embodiments:

1. A method for creating a patient care plan for a target patient, the method including: receiving inputs (66, 68, 70, 72) related to one or more social services (54) and one or more medical services (52) that are each associated with target patient data; selecting one or more social and medical services (52, 54) based on a target assessment; calculating a net care benefit for each of the selected services (52, 54); creating a patient care plan (106) from outcomes (92) including the selected services (52, 54) with the highest net care benefit.
2. The method according to embodiment 1, further including: selecting the most cost-effective services for the patient care plan (106).
3. The method, according to either embodiment 1 and 2, wherein calculating the net care benefit for each of the selected services (52, 54) includes: calculating the net care benefit as a function of ability-to-pay, including indicating at least one service (52, 54) that results in a high net care benefit over a target's ability-to-pay.
4. The method, according to embodiments 1-3, further including: estimating at least one of medical benefits, resource consumption, and service costs for each of the selected medical and social services (52, 54).
5. The method, according to embodiments 1-4, wherein estimation of at least one of the medical effects, resource consumption, and service costs for each of the selected services (52, 54) further includes: retrieving historical target data including medical outcomes and costs for a population of targets who underwent each service (52, 54).
6. The method, according to embodiments 1-5, further including: translating the inputs (66, 68, 70, 72) into one or more profiles (78); and generating a general service plan (88) from the one or more profiles (78) that corresponds to the selected medical and social services (52, 54) of the population of target patients, including one or more selected medical and social services outcomes (94, 96).
7. The method, according to embodiments 1-6, further including: translating the general service plan (88) into one or more statuses (80); generating a personalized service plan (90) from one or more status vector arrays (80) for the target patient, including one or more medical and social services outcomes (94, 96).
8. The method, according to embodiments 1-7, further including: continuously monitoring a profile (78) and a status (80) of the target during implementation of the patient care plan; and updating the patient care plan (106) to achieve the highest net care benefit.
9. The method, according to embodiments 1-8, further including: translating each of the selected medical and social services (52, 54) into a service status (100);
creating the patient care plan (106) from scores (104) calculated from the service statuses of the medical services (52), the social services (54), a reference status (102) of the medical services (52), and the social services (54) of the population of target patients.
10. The method, according to embodiments 1-7, further including: classifying each of the scores (104).
11. The method according to embodiments 1-9, wherein: the medical services (52) are selected from the group, including: inpatient clinic services, outpatient clinic services, primary care, rehabilitation services, mental medical services, and palliative care, and the social services; (54) are selected from a group including financial assistance services, housing services, personal care services, psychological support services, patient group services, social activities services, dietary support services, employment services, income services, legal services, transportation and mobility services, care management services, home care services, information and assistance services, long-term care services, nutrition and meals services, respite services, senior services, volunteer and intergenerational services, and wellness and well-being services.
12. A patient care plan system, including: one or more processors (16) programmed to perform the method according to claims 1-11, using a display (12) for inputs (66, 68, 70, 72) and the created patient care plan (106).
13. The patient care plan system, according to embodiment 12, wherein at least one processor (16) is further programmed to create a patient care plan (106) with the highest net care benefit from each of the selected services (52, 54); and wherein the medical services (52) are selected from the group, including: inpatient clinic services, specialized outpatient clinic services, rehabilitation services, mental medical services, and palliative care; with social services (54) that are selected from the group including employment services, income services, legal services, transportation and mobility services, care management services, home care services, information and assistance services, long-term care services, nutrition and meals services, respite services, senior services, volunteer and intergenerational services, and wellness and well-being services.
14. The patient care plan system, according to embodiments 12 and 13, wherein the one or more processors (16) are further programmed to: translate the inputs (66, 68, 70, 72) into one or more profiles (78); generate a general service plan (88) from the one or more profiles (78) that corresponds to the selected medical and social services (52, 54) of the population of target patients, including one or more medical and social services outcomes (94, 96); translate the general service plan (88) into one or more statuses (80); generate a personalized service plan (90) from the one or more statuses (80) for the target, including one or more medical and social services outcomes (94, 96).
15. The patient care plan system, according to embodiments 12-14, wherein the one or more processors (16) are further programmed to: continuously monitor a profile (78) and the status (80) of the target patient during implementation of the patient care plan (106); and update the patient care plan (106) to achieve the highest net care benefit.
16. The patient care plan system, according to embodiments 12-15, wherein the one or more processors (16) are further programmed to: translate each of the selected medical and social services (52, 54) into a service status (100); and create the patient care plan (106) from scores (104) calculated from the service statuses (102) of the medical services (52), the social services (54) and reference status (102) of the medical services (52), and the social services (54) of the population of target patients.
17. A patient care plan system (10) means (16) to perform any of the methods according to embodiments 1-11.
18. The patient care plan system according to embodiment 17, wherein the means (16) further creates a patient care plan (106) with the highest net care benefit from each of the selected services (52, 54), and wherein the medical services (52) are selected from the group including: inpatient clinic services, specialized outpatient clinic services, rehabilitation services, mental medical services, palliative care, and social services (54) are selected from the group, including employment services, income services, legal services, transportation and mobility services, care management services, home care services, information and assistance services, long-term care services, nutrition and meals services, respite services, senior services, volunteer and intergenerational services, and wellness and well-being services.
19. The patient care plan system according to either embodiment 17 or 18, wherein the means (16) further translates the inputs (66, 68, 70, 72) into one or more profile vector arrays (78); generates a general service plan (88) from the one or more profile vector arrays (78) that corresponds to the selected medical and social services (52, 54) of the population of targets; including one or more medical and social service outcomes (94, 96), translate the general service plan (88) into one or more status vector arrays (80), generate a personalized service plan (90) from the one or more status vector arrays (80) for the target, including one or more medical and social service outcomes (94, 96).
20. The patient care plan system according to embodiments 17-19, wherein the means (16) is further programmed to: translate each of the medical and social services (52, 54) into a service status (100), create the patient care plan (106) from scores (104) calculated from the service statuses (100) of the medical services (52) and the social services (54), a reference status (102) of the medical services (52), and the social services (54) of the population of target patients.

What is claimed is:

1. A system for classifying user files based on historical data on medical and social results using artificial neural networks comprising multiple layers of inter-connected nodes that pass data according to computational rules from input layers to output layers using common weights while working in tandem on two different input vectors to compute comparable output vectors, the system comprising:
cloud-based storage circuitry configured to store an artificial neural network, wherein the artificial neural network is trained to predict file processing programs based on historical medical and socials results for a plurality of user files, and wherein the historical medical and social results are represented in respective vector arrays;
cloud-based storage circuitry configured to:
training the artificial neural network to classify labeled medical profile vector arrays into corresponding general medical service programs, wherein the general medical service programs each have a target medical result; and
training the artificial neural network to classify labeled social profile vector arrays into corresponding general social service programs, wherein the general social service programs each have a target social result;
receive a first vector array, wherein the first vector array represents results of medical services applied in a file;
receive a second vector array, wherein the second vector array represents results of social services applied in the file;
input the first vector array and the second vector array into the artificial neural network, wherein the artificial neural network uses representation learning to match the first vector array and the second vector array to the historical medical and socials results for the plurality of files; and
cloud-based input/output circuitry configured to generate for display, on a local device, receive a recommendation, from the artificial neural network, for a file processing program, wherein the recommendation comprises a combination of a selected general medical service program and a general social service program.

2. A method of classifying user files based on historical data on medical and social results using artificial neural networks comprising multiple layers of inter-connected nodes that pass data according to computational rules from input layers to output layers using common weights while working in tandem on two different input vectors to compute comparable output vectors, the method comprising:
receiving, using control circuitry, a first vector array, wherein the first vector array represents results of medical services applied in a file;
receiving, using control circuitry, a second vector array, wherein the second vector array represents results of social services applied in the file;
inputting, using the control circuitry, the first vector array and the second vector array into an artificial neural network, wherein the artificial neural network is trained to predict file processing programs based on historical medical and socials results for a plurality of files, and wherein the historical medical and social results are represented in respective vector arrays; and
receiving, using the control circuitry, a recommendation, from the artificial neural network, for a file processing program.

3. The method of claim 2, wherein the file processing program comprises a program with a highest net processing benefit from medical services and social services, wherein the medical services are selected from a group including in-file clinic services, specialized out-file clinic services, rehabilitation services, mental medical services, and palliative processing, and wherein the social services are selected from a group including employment services, income services, legal services, transportation and mobility services, processing management services, home processing services, information and assistance services, long-term processing services, nutrition and meals services, respite services, senior services, volunteer and intergenerational services, and wellness and well-being services.

4. The method of claim 2, further comprising
translating the results of the medical services applied to the file into the first vector array; and
translating the results of the social services applied to the file into the second vector array.

5. The method of claim 2, wherein training the artificial neural network to predict the successful file processing programs based on historical medical and socials results for the plurality of files comprises:
training the artificial neural network to classify labeled file medical profile vector arrays into corresponding general medical service programs, wherein the general medical service programs each have a target medical result; and
training the artificial neural network to classify labeled file social profile vector arrays into corresponding general social service programs, wherein the general social service programs each have a target social result.

6. The method of claim 5, wherein the recommendation, from the artificial neural network, for the file processing program comprises a combination of a selected general medical service program and a general social service program.

7. The method of claim 2, wherein the successful file processing programs are selected based on statistical models of successful processing programs based on the historical medical and socials results for the plurality of files.

8. The method of claim 2, further comprising:
continuously monitoring a profile and a status of the file during implementation of the file processing program;
generating a new first vector array based on the implementation; and
generating a new second vector array based on the implementation.

9. The method of claim 8, further comprising:
receiving the new first vector array, wherein the first vector array represents results of medical services applied to the file as a result of the implementation;
receiving the new second vector array, wherein the second vector array represents results of social services applied to the file as a result of the implementation;
inputting the new first vector array and the new second vector array into the artificial neural network; and
receiving a new recommendation, from the artificial neural network, for a new file processing program.

10. The method of claim 9, wherein the artificial neural network is a Bayesian classifier.

11. The method of claim 9, wherein the artificial neural network uses representation learning to match the first vector array and the second vector array to the historical medical and socials results for the plurality of files.

12. A non-transitory computer-readable medium for classifying user files based historical data on medical and social results using artificial neural networks comprising multiple layers of inter-connected nodes that pass data according to computational rules from input layers to output layers using common weights while working in tandem on two different input vectors to compute comparable output vectors comprising instructions that, when executed by one or more processors, cause operations comprising:
receiving a first vector array, wherein the first vector array represents results of medical services applied in a file;
receiving a second vector array, wherein the second vector array represents results of social services applied in the file;
inputting the first vector array and the second vector array into an artificial neural network, wherein the artificial neural network is trained to predict file processing programs based on historical medical and socials results for a plurality of files, and wherein the historical medical and social results are represented in respective vector arrays; and
receiving a recommendation, from the artificial neural network, for a file processing program.

13. The non-transitory computer-readable media of claim 12, wherein the file processing program comprises a program with a highest net processing benefit from medical services and social services, wherein the medical services are selected from a group including in-file clinic services, specialized out-file clinic services, rehabilitation services, mental medical services, and palliative processing, and wherein the social services are selected from a group including employment services, income services, legal services, transportation and mobility services, processing management services, home processing services, information and assistance services, long-term processing services, nutrition and meals services, respite services, senior services, volunteer and intergenerational services, and wellness and well-being services.

14. The non-transitory computer-readable media of claim 12, further comprising
translating the results of the medical services applied to the file into the first vector array; and
translating the results of the social services applied to the file into the second vector array.

15. The non-transitory computer-readable media of claim 12, wherein training the artificial neural network to predict the successful file processing programs based on historical medical and socials results for the plurality of files comprises:
training the artificial neural network to classify labeled file medical profile vector arrays into corresponding general medical service programs, wherein the general medical service programs each have a target medical result; and
training the artificial neural network to classify labeled file social profile vector arrays into corresponding general social service programs, wherein the general social service programs each have a target social result.

16. The non-transitory computer-readable media of claim 15, wherein the recommendation, from the artificial neural network, for the file processing program comprises a combination of a selected general medical service program and a general social service program.

17. The non-transitory computer-readable media of claim 12, wherein the successful file processing programs are selected based on statistical models of successful processing programs based on the historical medical and socials results for the plurality of files.

18. The non-transitory computer-readable media of claim 12, further comprising:
continuously monitoring a profile and a status of the file during implementation of the file processing program;
generating a new first vector array based on the implementation; and
generating a new second vector array based on the implementation.

19. The non-transitory computer-readable media of claim 18, further comprising:
receiving the new first vector array, wherein the first vector array represents results of medical services applied to the file as a result of the implementation;
receiving the new second vector array, wherein the second vector array represents results of social services applied to the file as a result of the implementation;
inputting the new first vector array and the new second vector array into the artificial neural network; and
receiving a new recommendation, from the artificial neural network, for a new file processing program.

20. The non-transitory computer-readable media of claim 19, wherein the artificial neural network is a Bayesian classifier.
